(19) United States
(12) Patent Application Publication
(10) Pub. No.: US 2003/0099621 A1
(43) Pub. Date: May 29, 2003

(54) Stem cell screening and transplantation therapy for HIV infection

(76) Inventors: Robert Chow, Arcadia, CA (US); Denis O. Rodgerson, Malibu, CA (US); Rubio R. Punzalan, Torrance, CA (US); Lawrence D. Petz, Tarzana, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/998,832

(22) Filed: Nov. 29, 2001

(51) Int. Cl. A61K 48/00; C12N 5/08; C12N 15/85

(52) U.S. Cl. 424/93.21; 435/372; 435/455

(57) ABSTRACT

This invention provides methods for preventing or treating any disease arising from HIV infection, including AIDS and AIDS-related complex (ARC). The method comprises screening a plurality of donors to identify stem cells with a beneficial gene or genes and then transplanting the therapeutic stem cells into a patient. In preferred embodiments, the beneficial gene encodes a polymorphism that renders cells refractory to HIV infection. The polymorphism may be for a gene that encodes a ligand of a receptor for HIV entry, a product of the HLA complex, or a receptor for HIV entry.

### BACKGROUND OF THE INVENTION

Human immunodeficiency virus (HIV) infection is most commonly treated with agents that interfere with viral replication, such as small molecule protease inhibitors, nucleoside analogues, and non-nucleoside reverse transcriptase inhibitors. These antiviral therapies have been relatively effective for reducing viral loads and restoring immune function. However, these drugs exhibit numerous side effects, require prolonged treatment that often induces drug resistance, and do not result in complete eradication of the virus from the body. As a consequence, a great deal of current research focuses on developing therapies which either enhance the ability of the immune system to neutralize HIV or interfere with the ability of the virus to infect immune cells. In particular, these therapies exploit the growing body of evidence that certain gene polymorphisms are associated with reduced susceptibility and disease progression (see, Roger et al. FASEB, 12:625-632 (1998); O'Brien et al. Hospital Practice, July 15 (1998); Hogan et al. Ann. Intern. Med., 134:978-996 (2001)).

Many of these beneficial polymorphisms are variants of receptors and ligands for receptors that mediate HIV entry into immune cells. Although human immunodeficiency virus type-1 (“HIV-1”) uses the T cell surface molecule CD4 as a primary receptor, successful viral entry into and infection of a cell has been found to require the presence of a second molecule, or “co-receptor” (see, Clapham and Weiss, Nature, 388:230-231 (1997)). Seven co-receptor molecules have been identified, each of which are members of, or related to, the family of chemokine receptors, which are G-protein coupled receptors having seven transmembrane domains. The chemokine receptor CCR5, which selectively binds RANTES, MIP-1alpha, and MIP-1beta, serves as a coreceptor for macrophage tropic-strains of HIV, whereas the stromal derived factor 1 (SDF-1) chemokine receptor CXCR4 is a coreceptor for T cell-tropic HIV strains. CCR3, CCR2b, and CCR1 serve as coreceptors for other less common HIV strains.

The first HIV resistance gene to be characterized was a polymorphism of the primary HIV coreceptor CCR5 (see, Dean et al. Science, 273:1856-1862 (1996); Liu et al. Cell, 86: 367-377 (1996)). A 32 basepair deletion of the CCR5 receptor (CCR5 delta 32) causes a frameshift mutation and deletion of the last three transmembrane domains. Individuals homozygous for such a deletion remain uninfected despite multiple sexual exposures to HIV. However, those heterozygous for this deletion are susceptible to infection, although progression to AIDS may be slowed. Another beneficial polymorphism is a point mutation at residue 303 of the CCR5 (CCR5m303), which creates a stop codon and deletion of the last five transmembrane domains and the cytoplasmic tail. This mutation confers resistance to HIV infection when associated with the CCR5 delta 32 mutation (see, Quillent et al. Lancet, 351: 14-18 (1998)). Polymorphisms (e.g., CCR5P1, CCR5 59029A and 59353C) in the promoter region of these coreceptors are also associated with rapid progression of the disease (see, Ometto et al. J. Infectious Disease, 183:814-818 (2001); Martin et al. Infectious Disease, 282:1907-1911 (1998); Clegg et al. AIDS, 14:103-108 (2000)).

Variants of other less utilized HIV coreceptors also appear to influence disease progression. A conservative point mutation of the CCR2 receptor. CCR2-64I, permits expression of the receptor but nevertheless delays disease progression (see, Smith et al. Science, 277:959-965 (1997)).

Polymorphisms of genes encoding ligands for the HIV coreceptors CCR5 and CXCR4 influence disease progression, but not susceptibility. For example, homozygosity for a point mutation in the 3′ untranslated region of a Stromal-derived Factor 1 alpha (SDF-1 alpha) delays disease progression in a recessive manner (see, Winkler et al. Science, 279:389-393 (1998)). It is hypothesized that the 3′A mutation upregulates the biosynthesis of SDF-1 alpha such that there is increased competition with HIV for CXCR4 receptors. A RANTES promoter polymorphism that increases RANTES expression is believed to function in a similar manner, but in this case by increasing competition with HIV for the CCR5 receptor (see, Liu et al. PNAS, 96:4581-4585 (1999)).

Finally, there is also evidence that HLA alleles influence HIV-1 disease progression. Animal studies demonstrate that resistance to murine AIDS maps to the H-2 complex, the mouse homologue of the HLA locus (see, Makino et al. J. Immunol., 144: 4347-4355 (1990)). The HLA complex contains three types of genes (class I, II, and III), all of which are involved in modulating the immune response. Class I (A, B, C, D, E, F,G) and class II (DM, DP, DQ, DR) molecules, commonly known as MHC genes, are both involved in antigen presentation to T cells. Class III HLA includes a variety of unrelated proteins, including the transporter for antigen processing (TAP), polypeptides of the proteasome, complement component factors (Bf, C2, C4), and tumor necrosis factors (TNF-alpha, TNF-beta).

Studies indicate that an individual's particular type of MHC class I and II molecules can influence disease progression. A study of pairs of HIV-1 infected hemophiliac brothers has demonstrated that sibling pairs sharing one or two HLA class II alleles exhibit similar rates of disease progression (see, Kroner et al. AIDS, 9:275-280 (1995)). A more recent study has found that HLA class I B*5701 is highly associated with restriction of viral replication in nonprogressors (see, Flores-Villanueva et al. PNAS, 98:5140-5145 (2001)). It is hypothesized that an enhanced ability of certain MHC proteins to associate with processed HIV-1 antigens allows certain individuals to mount a highly effective CD8 lymphocyte response against the virus.

Another polymorphism that influences HIV disease progression is IL10-5′A, a variant of the promoter region for interleukin-10 (IL-10). This polymorphism reduces IL10 production and is associated with rapid progression of AIDS in both homozygotes and heterozygotes (see, Shin et al. PNAS, 97:14467-72 (2000)). IL-10 is known to inhibit macrophage, T-lymphocyte, and HIV replication. Presumably, promoter mutations which increase IL-10 levels would slow progression of AIDS.

The discovery that certain polymorphisms confer resistance to HIV has led to the proposal of therapies which repopulate the immune system with cells more capable of resisting infection and/or more capable of neutralizing the virus. By preventing de novo infection of cells, such therapy can eliminate the need for prolonged treatment with inhibitors of viral replication. Furthermore, the specific nature of such therapies should reduce side effects.

WO 99/23253 and U.S. Pat. No. 6,153,431 describe vectors that can be used to express beneficial polymorphisms in existing lymphocytes or stem cells, suggesting the replacement of infected cells with transduced cells. Other publications generally propose replacement of non-HIV resistant, infected cells with cells from donors expressing HIV resistance genes.

However, transducing circulating T lymphocytes with disease resistance polymorphisms is problematic, since these cells are so widely disseminated that is it is difficult to reach all target cells using current vector delivery systems. Furthermore, in vitro genetic engineering of stem cells and gene therapy with such cells can also be problematic. It is difficult to cultivate and transduce stem cells in vitro. Beneficial genes may not be expressed at sufficiently high levels to be effective, genes allowing infection by HIV may not be effectively “knocked out” using present methods, and transduction may affect subsequent differentiation into cells of the immune system. Finally, infusions of stem cells from donors, whether in vitro engineered or not, are preferably performed after matching of HLA phenotypes. Differences between the donor and the recipient can cause rejection of the transplant or even worse, the immune cells of the donor tissue may attack the tissues of the host (graft-versus-host disease). Current methods do not allow rapid and efficient identification of cells expressing both the desired disease resistance genes and HLA phenotype.

Thus there remains a need for a method for treating HIV infection that effectively renders immune cells refractory to HIV infection and/or enhances the ability of the immune system to neutralize the virus with a reduced risk of immunologic incompatibility. This invention fulfills this and other needs.

### BRIEF SUMMARY OF THE INVENTION

In one embodiment, this invention provides methods for preventing or treating any disease arising from HIV infection, including AIDS and AIDS-related complex (ARC). The method comprises screening a plurality of cells from donors to identify persons with a beneficial gene and then transplanting the stem cells into a patients with HIV infection (or at risk for HIV infection). Advantageously, the method renders immune cells refractory to HIV infection and/or preserves or enhances the ability of the immune system to neutralize the virus with a reduced risk of immunologic incompatibility.

In one aspect, this invention provides a method for preventing or treating HIV infection. The method involves: a) screening of cells from a plurality of donors to identify donors having a beneficial gene(s), and b) transplanting stem cells containing the beneficial gene(s) into patients with HIV infection. Preferably, the beneficial gene(s) is a polymorphism of a gene(s) encoding a protein(s) expressed by immune cells. The beneficial gene(s) may be one that reduces the ability of HIV to infect an immune cell or one that can enhance the ability of an immune cell to neutralize the virus through immune reconstitution.

In certain embodiments, the beneficial gene is a polymorphism of a ligand for HIV entry, including, but not limited to, the 3′A polymorphism of SDF-1 alpha or a promoter polymorphism of RANTES that increases expression levels. In another embodiment, the polymorphism is of a gene in the HLA complex, which encodes MHC class I molecules, MHC class II molecules, TNF, and complement.

In yet another embodiment, the beneficial gene is a polymorphism of one of the receptors or coreceptors for HIV entry including, but not limited to, CD4, CXCR4, CCR2, and CCR5. These polymorphisms include, but are not limited to, CCR2-64I, a 32 basepair deletion in the coding region of CCR5, CCR5m303, and a polymorphism in the promoter region of CCR5.

In a preferred embodiment, the cells screened in this invention are obtained from embryos, marrow, peripheral blood, placental blood, umbilical cord blood, adipose tissue, or any other potential source of stem cells.

In certain embodiments, the cells are screened for a beneficial gene by detection or identification of the protein product (e.g., immunological assay) or any other protein assay. In other embodiments, the beneficial gene is detected using a hybridization-based assay, a sequencing assay, a functional assay, or other assay.

In a second aspect, the method described above further comprises ex vivo (in vitro) and/or in vivo expansion of the therapeutic stem cell unit.

In a third aspect, the method further comprises identification of the HLA genotype and/or phenotype of the therapeutic stem cell. The HLA genotype can optionally be determined via a high-throughput method using allele-specific primers and HLA locus-specific capture oligonucleotides immobilized on a solid phase.

In a fourth aspect, the method further comprises treatment of said stem cell to express a non-native HLA protein or to inhibit expression of the native HLA protein.

Other advantages, aspects, and embodiments of this invention will become apparent upon reading the following description.

### DEFINITIONS

The term “beneficial gene” as described herein refers to any gene which provides increased resistance to a disease, any gene which slows or reduces the progression of a disease, or any gene which is beneficial to research. Genes beneficial to research includes those that could be used to produce mammalian models of various diseases.

The term “cells from a donor” refers to any population of cells that contains stem cells and is extractable from a donor. Typical sources of such cells include embryos, bone marrow, peripheral blood, umbilical cord blood, placental blood, adipose tissue, and any other tissue in which stem cells reside.

The term “immune cell” as used herein refers to any cell which plays a role in the body's defense against pathogens. The primary immune cell targets of HIV are macrophages and T lymphocytes.

The term “polymorphism” as described herein refers to a variant of the sequence of a particular gene. This includes differences in genotypes ranging in size from a single nucleotide site to a large nucleotide sequence visible at a chromosomal level.

The term “in vitro expansion” refers to the cultivation of mammalian cells in the laboratory. Such cells can be extracted from a mammal and additional quantities of cells generated by cultivation in the appropriate environment. If possible, stable cell lines are established to allow for continued propagation of cells.

The term “stem cell” refers to any cells that have the ability to divide for indefinite periods of time and to give rise to specialized cells. Stem cells emanate from all germinal layers (ectoderm, mesoderm, and endoderm). Typical sources of stem cells include embryos, bone marrow, peripheral blood, umbilical cord blood, placental blood, and adipose tissue. Stem cells can be pluripotent, meaning that they are capable of generating most tissues on an organism. For example, pluripotent stem cells can give arise to cells of the skin, liver, blood, muscle, bone, etc. In contrast, multipotent or adult stem cells can only give arise to limited types of cells. For example, the hematopoietic stem cell can only give arise to cells of the lymphoid and myeloid lineages.

The term “HIV” or “human immunodeficiency virus” refers to HIV-1, HIV-2, and any other strains of the virus which contribute to the development of AIDS or AIDS-related complex (ARC).

The term “HIV infection” as used herein refers to any of the spectrum of conditions associated with HIV infection, ranging from asymptomatic seropositivity, through AIDS-related complex (ARC), to acquired immunodeficiency syndrome (AIDS).

The term “acquired immunodeficiency syndrome” or “AIDS” as described herein refers to defects in cellular immunity associated with a infection with HIV, low CD4 positive T lymphocyte counts, and increased susceptibility to opportunistic infections and malignant neoplasms.

The term “HLA complex” as used herein refers to the collection of genes on a chromosome that encode MHC class I, class II, and class III molecules. The “MHC class I” molecules are glycoproteins which present antigens to T helper cells, whereas “MHC class II” molecules present antigens to cytotoxic T cells. “MHC class III” molecules are secreted proteins, such as proteins of the complement pathway and tumor necrosis factor (TNF).

The term “TNF” or “tumor necrosis factor” as used herein refers to the two related cytokines produced by macrophages (TNF-alpha) and some T cells (TNF-beta). These factors are cytotoxic to tumor cells and play a role in the inflammatory response.

The term “complement” as used herein refers to any of the group of serum proteins which form the membrane-attack complex, a complex which mediates cell lysis.

### BRIEF DESCRIPTION OF THE DRAWINGS

FIG. 1 illustrates a flow diagram for one embodiment of the collection and use of stem cells in the treatment of HIV/AIDS patients.

### DETAILED DESCRIPTION

I. Introduction

This invention provides, inter alia, a method for preventing or treating any disease arising from HIV infection, including AIDS and AIDS-related complex (ARC). The method comprises screening a plurality of cells to identify stem cells with a beneficial gene and then transplanting the stem cells into a patient. In certain embodiments, potential donors are first screened for beneficial mutations and then stem cells are extracted from these donors for transplantation.

In preferred embodiments, the beneficial gene is a polymorphism of a gene that renders immune cells refractory to HIV infection or a gene that enhances the ability of immune cells to neutralize the virus. Preferably, the gene encodes a ligand of a receptor for HIV entry, a gene of the HLA complex, or a receptor for HIV entry. The cells screened in this invention are derived from sources which contain stem cells, including, but not limited to, embryos, the umbilical cord, the placenta, marrow, peripheral blood, and adipose tissue. They are screened, preferably in a high-throughput manner, for the beneficial gene using any method that detects the polymorphism of the gene and/or the protein variant. The therapeutic stem cells are optionally HLA-typed using high-throughput methods before transplantation into matched recipients.

This invention also provides a method for producing a disease model by screening a plurality of cells from donors to identify stem cells with a gene that induces disease and transplanting the identified stem cells into a mammal, such as a mouse, to induce disease. This mammal can be used to test potential therapies and to elucidate the mechanism of the disease.

II. Beneficial Genes of this Invention

Beneficial genes of this invention can be beneficial for fighting HIV infection or beneficial for research. Genes beneficial for research include those that can be used to induce disease in mammals to produce disease models.

In preferred embodiments, the beneficial genes are beneficial for fighting HIV infection. The beneficial genes can either render immune cells resistant to HIV infection, or enable the immune cells to more effectively neutralize the virus via immune reconstitution. These beneficial genes can be polymorphisms of genes encoding proteins expressed by immune cells, genes advantageous for fighting infection that are not expressed in the patient, or any other genes that enhance the ability of immune cells to resist HIV infection and/or neutralize the virus. Such genes are described in immunology reference texts (see, Kuby et al. Immunology, 3rd. ed. W. H. Freeman & Co.). Exemplary polymorphisms that confer decreased susceptibility to HIV and reduced disease progression are described in several reviews (see, Roger et al. FASEB, 12:625-632 (1998); O'Brien et al. Hospital Practice, Jul. 15 (1998); Hogan et al. Ann. Intern. Med., 134:978-996 (2001); Medscape HIV/AIDS update 2000; Michael, Current Opin. Immunol., 11:466-474 (1999)).

In one embodiment of this invention, the beneficial gene renders immune cells resistant to HIV infection. This gene can be a polymorphism of a gene encoding any receptor that facilitates entry of HIV into the immune cells. Receptors that mediate HIV entry include the primary cellular receptor CD4, as well as coreceptors, including, but not limited to, CXCR4, CCR5, CCR2b, CCR3, and CCR1. Suitable polymorphisms include those that interfere with expression of the receptor at the cell surface (e.g., CCR5 delta 32, CCR5m303); ones that produce a receptor that is expressed, but unable to facilitate entry of the HIV virus (e.g., CCR2-64I); and promoter polymorphisms that regulate coreceptor expression levels. The beneficial gene can also be a polymorphism of the promoter region that increases expression of any ligand for a HIV receptor. The increased levels of ligand compete with HIV and thus reduce the ability of HIV to bind to the appropriate receptor. Ligands for HIV receptors include RANTES, MIP-1 alpha, MIP-1 beta, SDF-1 alpha, and SDF-1 beta. Polymorphisms which increase expression levels include the RANTES-35G promoter variant and the SDF-1 alpha 3′A variant.

In another embodiment of this invention, the beneficial gene enables the immune cells to more effectively neutralize the virus. The beneficial gene can encode any protein that allows an individual to mount a more effective immune response against pathogens. In certain embodiments, the gene is a polymorphism of a promoter region for a product, such as IL-10, which inhibits HIV replication. In other embodiments, the gene is in the HLA locus. The gene can encode a MHC class I molecule, a MHC class II molecule, or a class III molecule associated with reduced susceptibility or reduced disease/progression. MHC class I alleles include B, C, and A gene products. MHC class II alleles include DP, DQ, and DR gene products. Class III molecules include complement and tumor necrosis factor. In certain preferred embodiments, the gene encodes the MHC class I molecule HLA B*5701.

III. Cell Populations to be Screened in the Method of this Invention

In one embodiment, the methods of this invention comprise screening a plurality of cells from donors to identify persons and cells with a beneficial gene. The population of cells to be screened should include stem cells. Preferably, the cell population is rich in stem cells. Stem cells emanate from all germinal layers (ectoderm, mesoderm and endoderm). Stem cell-rich populations can be obtained from existing cell lines or isolated from banked collections of stem cell sources. Typical sources of stem cells include, embryos, marrow, peripheral blood, placental blood, umbilical cord blood, adipose tissue and others. Harvesting, enrichment, and cryopreservation techniques are described in Bone Marrow and Stem Cell Processing : A Manual of Current Techniques Ellen M. Areman (Editor), H. Joachim Deeg, Ronald A. Sacher (Editor) Philadelphia (1992).

Preferably, the cells to be screened are obtained from sources which allow for rapid and easy collection of a cells from a variety of unrelated individuals. Screening of cells from unrelated individuals provides the greatest chance of identifying cells with both the beneficial gene and a compatible HLA genotype. The therapeutic stem cells of this invention can be any type of stem cell which is capable of differentiating into cells that are infected by HIV, cells that can modulate the immune response against HIV, cells that mediate the immune response against HIV or cells that can reduce progression of AIDS. Such stem cells include, but are not limited to, embryonic stem cells, which can form many different types of stem cells, and hematopoietic stem cells, which can form blood and immuune cells, and other cells. Another potential source of stem cells is adipose tissue.

IV. Screening Stem cell-rich Cell Populations of this Invention

Cells are typically screened to identify cells with a beneficial gene. In certain embodiments, the cells are also screened for a HLA genotype compatible with the patient. The samples used for screening may consist of cells taken directly from a donor, or from cell lines established from donor cells. The cells can be screened simultaneously for beneficial genes and HLA genotype, or screened sequentially. Those cells with a beneficial gene and an appropriate HLA genotype are then prepared for transplantation into a patient.

A. Screening for Beneficial Genes

Cells are typically screened for beneficial genes using standard methods known to those of skill in the art for detection of particular nucleic acid sequences or proteins. In order to allow for rapid identification of therapeutic stem cell units expressing a beneficial gene, the methods are preferably ones which can be used in a high-throughput manner. Each cell sample from a donor may be screened for a variety of beneficial genes simultaneously. Alternatively, multiple samples are screened for presence of a particular beneficial gene.

In some embodiments, the cells are screened for beneficial genes using standard nucleic acid hybridization-based methods. In a particularly preferred embodiment, the cells are screened using a modification of the high-throughput HLA-typing methods described in U.S. patent application Ser. No. 09/747,391, filed Dec. 20, 2000, herein incorporated by reference. Briefly, the method comprises: a) isolating template nucleic acid from the donor cells; b) amplifying the template nucleic acid; c) hybridizing the template nucleic acid with an immobilized array of capture oligo nucleotides, each having a known nucleic acid sequence of the beneficial genes being screened for; and d) determining the particular capture oligonucleotide to which the template nucleic acid hybridizes, thereby determining whether the cells have a beneficial gene.

In other embodiments, the cells are screened for beneficial genes using any standard immunological methods suitable for detecting the protein product of a beneficial gene, i.e., Western blotting, standard immunoassays, and flow cytometry. A general overview of immunoassay technology can be found in Harlow & Lane, Antibodies: A Laboratory Manual (1988). The proteins expressed by beneficial genes of the invention can be detected and/or quantified using any of a number of well recognized immunological binding assays (see, e.g., U.S. Pat. Nos. 4,366,241; 4,376,110; 4,517,288; and 4,837,168). For a review of general immunoassays, see Methods in Cell Biology: Antibodies in Cell Biology, volume 37 (Asai, ed. 1993) and Basic and Clinical Immunology (Stites & Terr, eds., 7^{th} ed. 1991). Immunological binding assays (or immunoassays) typically use an antibody that specifically binds to a protein or antigen of choice (in this case the protein expressed by the beneficial gene or an antigenic subsequence thereof). The antibody may be produced by any of a number of means well known to those of skill in the art.

Under conditions when the beneficial gene encodes a protein that is not expressed, the cells may be screened for absence of protein.

B. Screening for HLA type

In certain embodiments, the cells containing beneficial genes are transplanted without HLA typing. In other embodiments, the cells are HLA typed to ensure compatibility with the recipient.

The HLA genotype of the cells can be determined by any number of means known to those of skill in the art. Preferably, the HLA genotype is determined using the high-throughput HLA typing method described in U.S. patent application Ser. No. 09/747,391, filed Dec. 20, 2000. Briefly, the method comprises: (a) isolating template nucleic acid from the cells; (b) amplifying the template nucleic acid to generate sufficient product for each allele of at least one gene locus to be determined; (c) hybridizing the template nucleic acid with an immobilized array of capture oligonucleotides, each having a known nucleic acid sequence of an HLA allele; and (d) determining the particular capture oligonucleotide to which the template nucleic acid hybridizes, thereby determining the genotype of the subject.

Other standard methods include serological and cellular typing (see, Terasaki and McClelland, Nature, 204:998 (1964)), restriction fragment length polymorphism (RFLP) analysis, hybridization of PCR amplified products with sequence-specific oligonucleotide probes (PCR-SSO) to distinguish between HLA alleles (see, Tiercy et al., Blood Review, 4: 9-15 (1990)), sequence-specific primer amplification (PCR-SSP) (see, Olerup and Zetterquist Tissue Antigens, 39: 225-235 (1992)), and Single-Stranded Conformational Polymorphism (SSCP).

V. Transplantation of Stem Cell-rich Cell Populations into Patients

After screening, cells expressing the desired beneficial gene and appropriate HLA genotype are selected and prepared for transplantation. If desired, the therapeutic stem cell units are expanded ex vivo (in vitro) using standard methods used to culture stem cells and maintain stable cell lines. Alternatively, these cells can be expanded in vivo. These cells can be used for future transplantation procedures. In certain embodiments the stem cell-rich cell populations are further enriched for stem cells prior to transplantation. Methods to select for stem cells are well known in the art. For example, samples can be enriched by tagging cell-surface markers of undifferentiated hematopoietic stem cells (e.g., CD34, CD59, Thyl, CD38 low, C-kit low, lin minus) with fluorescently labeled monoclonal antibodies and sorting via fluorescence-activated cell sorting (FACS). In other embodiments, a sample of the stem cell-rich population is transplanted without further enrichment.

Typically, the normal stem cell population (which ultimately produces the lymphocytes susceptible to viral replication) is eliminated or reduced prior to transplantation of the therapeutic stem cell units. Chemotherapy, radiation, or the techniques described in U.S. Pat. No. 6,217,867 are used to condition the bone marrow for appropriate engraftment of the transplant. Finally, therapeutic stem cell units expressing the beneficial gene are transplanted into the patient using standard methods.

VI. Therapeutic Applications

Preferably, the methods of this invention can be used to treat or prevent any disease or condition that arises from HIV infection, such as AIDS and ARC. It should be recognized that methods of this invention can easily be practiced in conjunction with existing antiviral therapies to effectively treat or prevent disease.

Factors and events which form a theoretical basis for the embodiments of the invention are discussed herein. However, this discussion is not in any way to be considered as binding or limiting on the present invention. Those of skill in the art will understand that the various embodiments of the invention may be practiced regardless of the model used to describe the theoretical underpinnings of the invention.

### EXAMPLES

The following examples are offered to illustrate, but not to limit the claimed invention.

Example 1

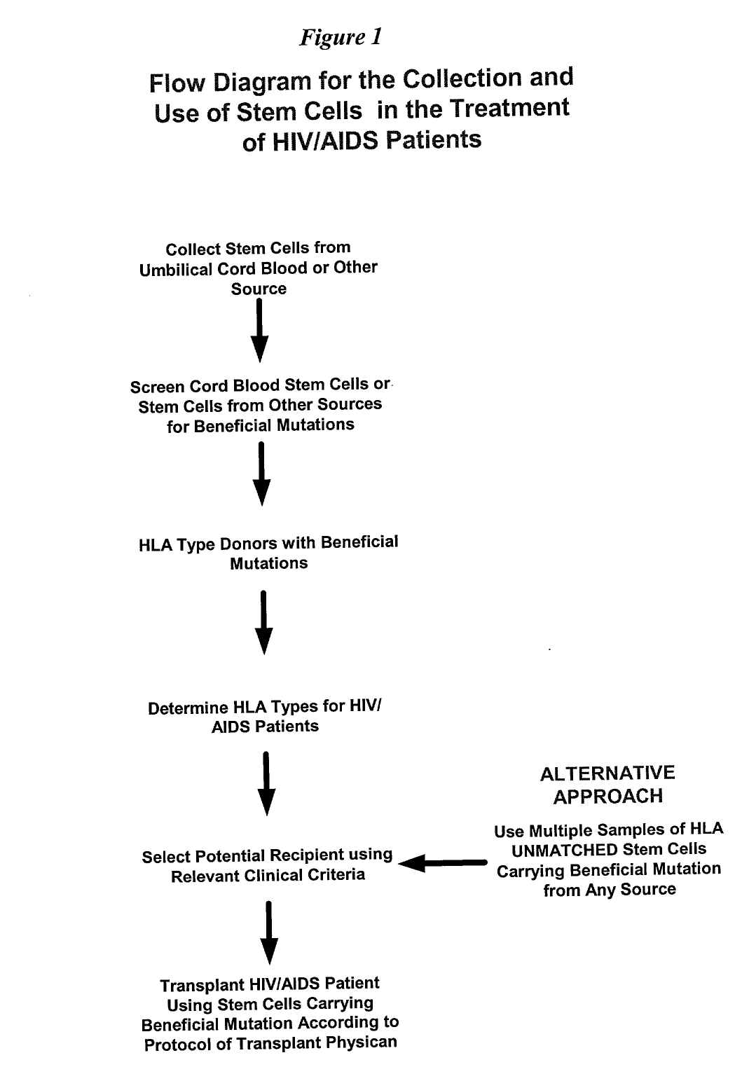

This example illustrates one embodiment of a method of this invention (FIG. 1). Stem cells are collected from umbilical cord blood or another suitable source and then screened for beneficial mutations. Alternatively, potential donors are screened for beneficial mutations using standard genotyping methods. Once stem cells and/or donors with beneficial mutations are identified, they are HLA-typed and matched with the HLA types of HIV/AIDS patients desiring treatment. Next, potential recipients are selected using relevant clinical criteria and the stem cells are transplanted according to standard stem cell transplantation protocols. In certain instances, stem cells are also transplanted into patients without HLA matching.

Example 2

This example illustrates the method of this invention when it is used to screen for the CCR5 delta 32 polymorphism. Immune cells from individuals homozygous for this deletion remain uninfected despite infection of the individual with HIV, presumably because the mutation prevents expression of the HIV coreceptor CCR5 at the cell surface.

Umbilical cord blood samples from unrelated infants are obtained from the Stemcyte umbilical blood cord bank. DNA is extracted from the enriched samples using the salt extraction method. The cells are first lysed and centrifuged. Then water is added and the sample is centrifuged again. The pellet is digested with Proteinase K. The DNA is then extracted by the addition of 6M Guanidine HCl and incubation at 70° C. for several minutes. The sample is centrifuged again and the supernatant is precipitated with cold 95% ethanol. The pellet is then dried and resuspended in the appropriate buffer.

The DNA samples are hybridized to an 96-well array, each target containing a nucleic acid sequence corresponding to that of the CCR5 delta 32 polymorphism. Images of the microarrays are collected using a CCD camera and analyzed to identify target elements associated with fluorescent signal. These elements indicate umbilical cord blood samples which express the CCR5 delta 32 polymorphism. Samples with cells containing the polymorphism are HLA-typed using the procedure described in U.S. patent application Ser. No. 09/747391. Samples with the polymorphism and the closest HLA match to the patient are selected and transfused intravenously into patients.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for preventing or treating HIV infection, said method comprising:
a) screening a plurality of cells to identify stem cells having a beneficial gene; and
b) transplanting said stem cells into a patient, thereby preventing or treating said HIV infection.

2. The method of claim 1, wherein said beneficial gene is a polymorphism of a gene encoding a protein expressed by immune cells.

3. The method of claim 1, wherein said beneficial gene is one which reduces the ability of HIV to infect an immune cell.

4. The method of claim 1, wherein said beneficial gene is one which enhances the ability of an immune cell to neutralize the virus through immune reconstitution.

5. The method of claim 2, wherein said protein is a ligand of a receptor for IV entry.

6. The method of claim 5, wherein said ligand is SDF-1 alpha and said polymorphism is SDF-1 alpha 3′A.

7. The method of claim 5, wherein said ligand is RANTES and said polymorphism is in the promoter region and increases expression levels.

8. The method of claim 2, wherein said protein is encoded by a gene in the HLA complex.

9. The method of claim 8, wherein said protein encoded by a gene in the HLA complex is selected from the group consisting of MHC class I molecule, MHC class II molecule, TNF, and complement.

10. The method of claim 2, wherein said protein is a receptor or coreceptor for HIV entry.

11. The method of claim 10, wherein said receptor for HIV entry is CD4.

12. The method of claim 10, wherein said coreceptor for HIV entry is CCR2.

13. The method of claim 12, wherein said polymorphism is CCR2-64I.

14. The method of claim 10, wherein said coreceptor for HIV entry is CCR5.

15. The method of claim 14, wherein said polymorphism is a 32 basepair deletion in the coding region.

16. The method of claim 14, wherein said polymorphism is CCR5m303.

17. The method of claim 14, wherein said polymorphism is in the promoter region of CCR5.

18. The method of claim 1, wherein said plurality of cells are obtained from the group consisting of embryos, marrow, peripheral blood, placental blood, umbilical cord blood, and adipose tissue.

19. The method of claim 1, further comprising in vitro or in vivo expansion of said stem cells.

20. The method of claim 1, wherein said method further comprises identification of the HLA genotype or phenotype of said stem cells.

21. The method of claim 20, wherein said identification of the HLA genotype is via a high-throughput method using allele-specific primers and HLA locus-specific capture oligonucleotides immobilized on a solid phase.

22. The method of claim 1, wherein said screening comprises identification of stem cells expressing the protein product of said beneficial gene.

23. The method of claim 22, wherein said protein product is detected or identified using an immunological assay.

24. The method of claim 1, wherein said screening comprises identification of stem cells with said beneficial gene.

25. The method of claim 24, wherein said beneficial gene is detected using a hybridization-based assay, a sequencing assay, or a functional assay.

26. The method of claim 1, further comprising treatment of said stem cells to express a non-native HLA protein or to inhibit expression of the native HLA protein.
